(19) United States
(12) Patent Application Publication
(10) Pub. No.: US 2015/0112278 A1
(43) Pub. Date: Apr. 23, 2015

(54) Systems and Methods for Needle for Subclavian Vein Penetration

(71) Applicant: Optima MDevice Technology Corporation, Andover, MA (US)

(72) Inventors: Subir Ray, Shenandoa, IA (US); Bireswar Chakraborty, Andover, MA (US); Kuowei Chang, Lexington, MA (US)

(73) Assignee: Optima MDevice Technology Corporation, Andover, MA (US)

(21) Appl. No.: 14/058,881

(22) Filed: Oct. 21, 2013

(51) Int. Cl. A61M 25/06; A61M 5/34; A61M 5/158; A61M 5/32

(52) U.S. Cl. CPC A61M 25/065; A61M 5/3286; A61M 5/3293; A61M 5/343; A61M 5/158; A61M 25/0606; A61M 2005/341
USPC 604/240; 604/272

(57) ABSTRACT

Systems and methods for placement of an introducer needle into the subclavian vein. A needle for subclavian vein penetration includes a straight needle portion, a bent needle portion, and a hub. The bent portion allows for needle placement with minimized chance of complications. The needle and hub form a lumen through which a guide wire can be placed. The bent needle portion has an inner diameter substantially similar to the inner diameter of the straight needle portion to reduce resistance due to movement of the guide wire.

### TECHNICAL FIELD

This application relates to surgical needles and, more particularly, to needles for insertion into the subclavian vein.

### BACKGROUND

As is now in the art, approximately five million surgical procedures per year require a needle to be advanced into the subclavian vein. Once inserted, the needle can be used to place a central venous catheter. In many of these procedures, a standard, straight needle is used. However, using a straight needle can result in complications. For example, the subclavian vein is very close to the lung. If the needle is inserted too far into the body, and/or at an incorrect angle, the needle could puncture the lung.

To reduce the chance of puncturing the lung, the needle should be inserted in a medial direction and not angled toward the anterior or posterior of the patient. In other words, with the patient in the decubitus position (i.e. lying on a table), the needle should be parallel to the floor—not angled up or down. However, the humerus bone and surrounding tissue can interfere with this positioning. When the shoulder is at rest or in a forward position, the anterior of the shoulder near the humeral head can cause the needle to be angled subcutaneously toward the posterior of the patient (i.e. toward the floor), increasing the chance of puncturing the lung. Central line subclavian vein cannulation has always been fraught with complications leading to pneumothorax, hemothorax, arterial and thoracic duct punctures, venous laceration, brachial plexus and other injuries. Judicious selection of introducer needle puncture point, angle, needle advancement direction, and subcutaneous needle path are required for gaining entry into the vein. The most important requirement is for the needle to advance very close to and in parallel with the chest surface.

A scapula wedge or rolled towels can be placed underneath the patient's neck and spine to move the shoulder back and out of the way of the needle. However, the scapula wedge or roiled towel can potentially injure or make the patient uncomfortable. In addition, even with the scapula wedge in place, the shoulder can still interfere with needle placement, especially in older patients whose shoulders are not flexible.

Shaped, curved and bent needles have been used for insertion and penetration into less-accessible body locations. These needles are exclusively used for fluid introduction, aspiration, and sample retraction. In fabrication of all these needles, there is no requirement for maintaining a suitable lumen to accommodate a smoothly sliding guide wire to pass through the needle. The applications of these shaped needles include administering a fluid or local anesthetic into a ligamentary tissue of an oral cavity, spinal intrathecal space, retinal blood vessel, eye, blood vessel, human face, ear canal, and many other body locations. None of these shaped needles are usable or are intended for placement of subclavian vein catheters.

### BRIEF DESCRIPTION OF THE DRAWINGS

The foregoing features may be more fully understood from the following description of the drawings. The drawings aid in explaining and understanding the disclosed technology. Since it is often impractical or impossible to illustrate and describe every possible embodiment, the provided figures depict one or more exemplary embodiments. Accordingly, the figures are not intended, to limit the scope of the invention. Like numbers in the figures denote like elements.

FIG. 1 is an anatomical drawing of the subclavian vein and a needle of the prior art.

FIG. 2 is an anatomical drawing of the clavicle area and needle placement of the prior art.

FIG. 3 is a schematic drawing of an embodiment of a bent introducer needle assembly.

FIG. 4 is a schematic drawing of another embodiment of a bent introducer needle assembly.

FIG. 5 is a schematic drawing of an embodiment of a bent introducer needle assembly having a hub extension.

FIG. 6 is a schematic drawing of an embodiment of another bent introducer needle assembly having hub extension.

FIG. 7 is an anatomical thawing of the clavicle are with needle placement.

FIG. 8 is a schematic drawing of an embodiment of a bent introducer needle assembly having at attached syringe.

FIG. 9 is a schematic drawing of an embodiment of a bent introducer needle assembly with a guide wire placed through the needle.

FIG. 10 is a schematic drawing of a straight needle shaft.

FIG. 11 is a side-view schematic drawing of a jig for bending a needle.

FIG. 12 is a cross-sectional view of the jig in FIG. 11

### SUMMARY

In an embodiment, a needle for subclavian center line catheter placement includes a proximal needle portion corresponding to a straight hub and formed of a rigid material. The straight hub has an outside diameter, a hollow interior, a length, a first end configured to be coupled to a syringe, and a second end. A needle shaft portion is formed of a high hardness metal, said needle shaft portion having a first straight end rigidly affixed to a portion of the hollow interior of the straight hub.

The first straight end of the needle shaft portion includes an embedded section having a length which extends at least partially through the hollow interior of the straight huh with at least a portion of the embedded section forming a friction fit within the straight hub portion. An exposed section of the needle shaft extends distally beyond the second end of the straight hub. The exposed section of the needle shaft portion has a bent shaft portion proximate to the second end of the straight hub. A straight shaft portion distal of the bent shaft portion forms an angle sufficient to allow the straight shaft portion of the needle to be inserted into a subclavian vein without a body of a patient interfering with movement or position of the hub.

The bent shaft portion has an interior diameter substantially similar to an inner diameter of the straight shaft portion.

### DETAILED DESCRIPTION

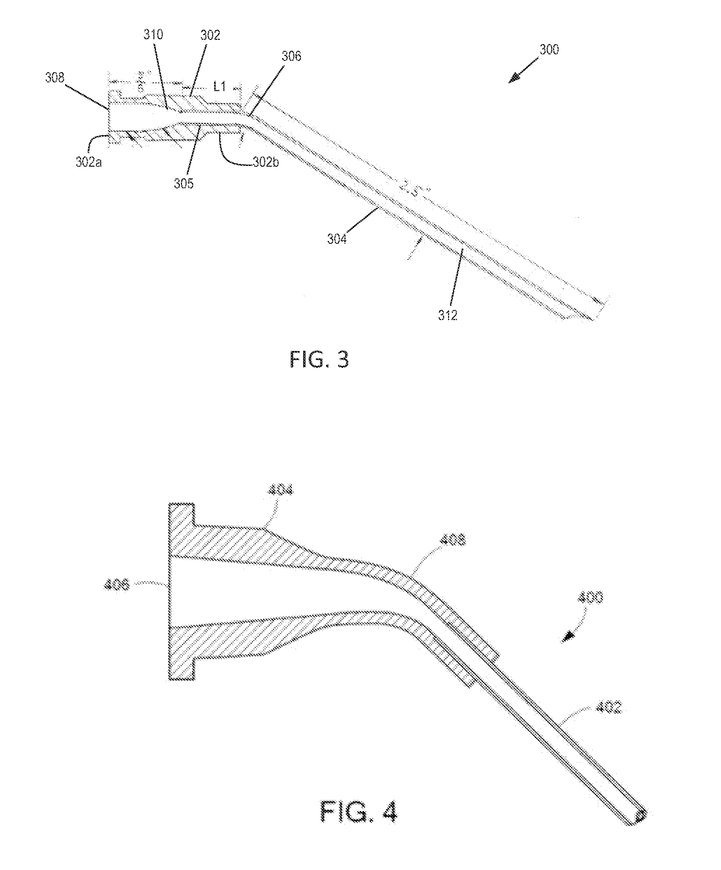

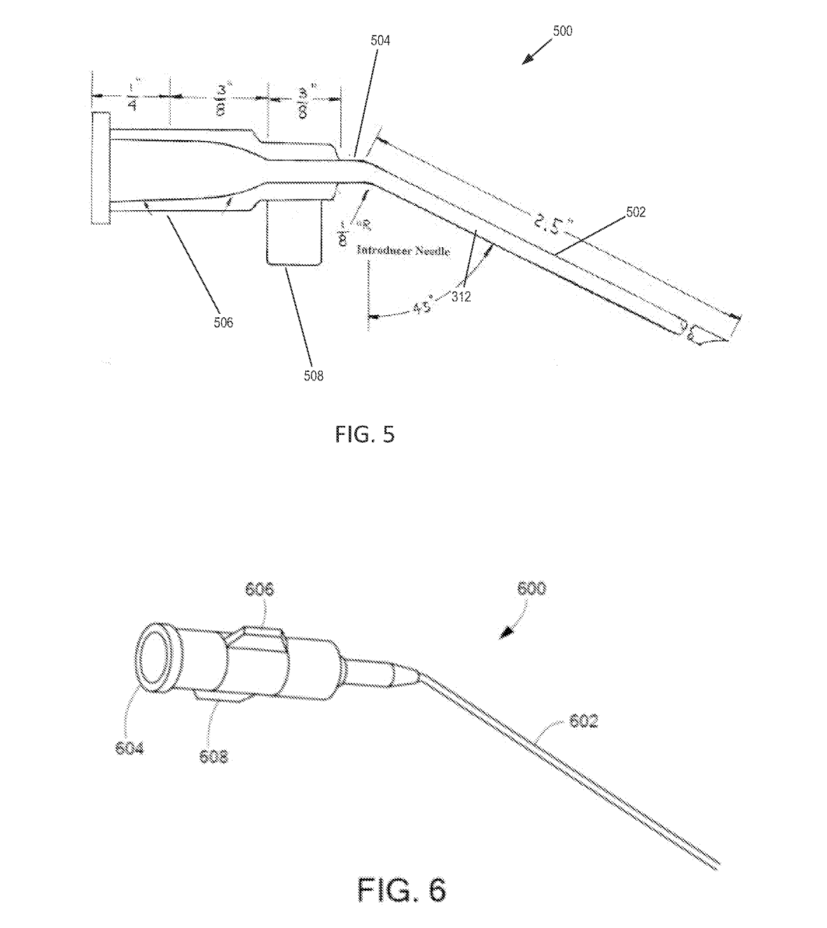

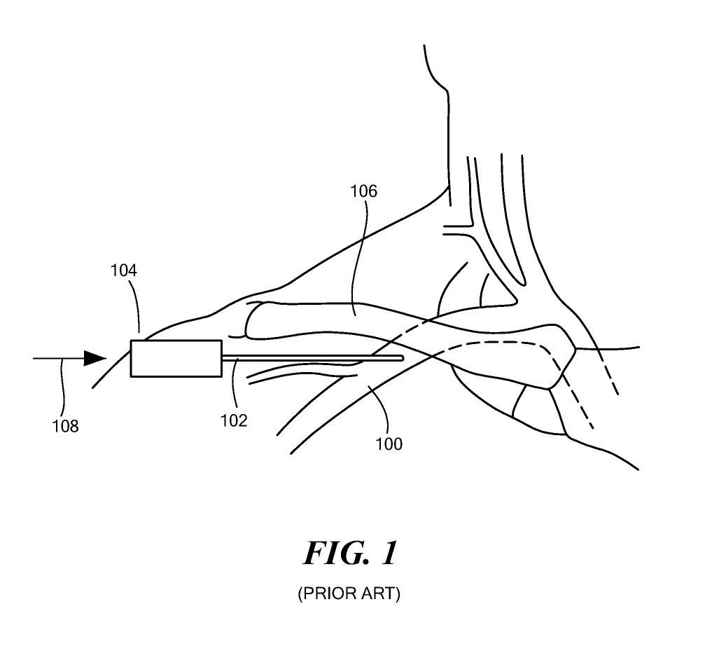

FIG. 1 is an anatomical diagram of a patient's shoulder showing insertion of a straight needle 102 of the prior art into the patient's subclavian vein. To access the subclavian vein 100, needle 102 is positioned on the anterior of the shoulder 104, inferior to the clavicle bone 106, and advanced medially (i.e. in the direction shown by arrow 108) until the needle enters the subclavian vein 100. Upon entry, the tip of needle 102 may be positioned underneath the clavicle 106.

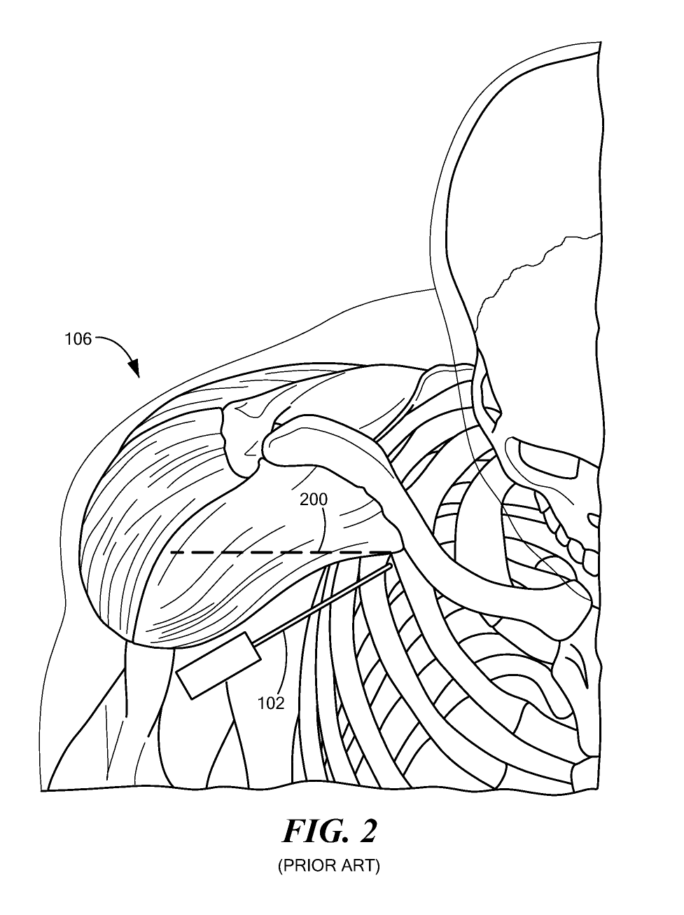

FIG. 2 is a top-view anatomical diagram of the patient's shoulder. Ideally, with the patient in a supine position, needle 102 should be inserted parallel to the floor (i.e. along the path illustrated by dotted line 200). However, due to the structure and position of the shoulder 106, the straight needle 102 must be angled subcutaneously inward toward the lung and ribcage—increasing the likelihood of complication by puncturing the lung, which lies just behind the clavicle and inside the rib cage.

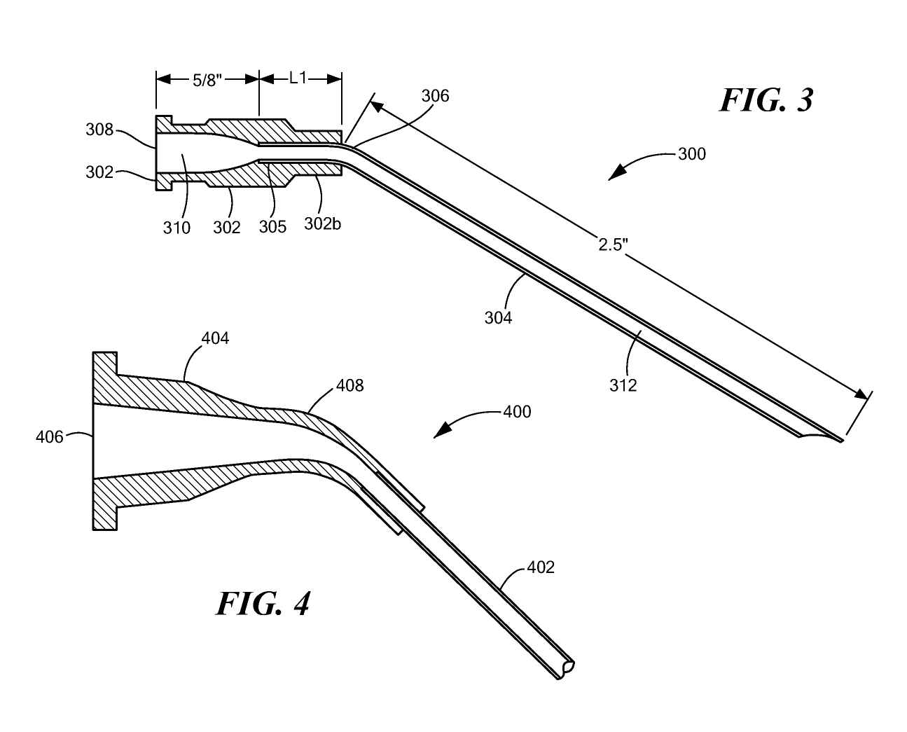

A method and system for the safe placement of subclavian vein catheter is described with reference to FIG. 3 and FIG. 4. The method and system are based on the use of an introducer needle assembly 300 (e.g. for subclavian center line catheter placement) having a bend or curve so that the portion of the needle that enters the patient's body is aligned at an angle to the syringe body axis and/or needle hub 302 (FIG. 3).

The needle assembly 300 includes a proximal needle portion corresponding to a hub 302 formed of a rigid material and having an outside diameter, a hollow interior, and a length, and having a first end 302a configured to be coupled to a syringe and a second end 302b. The hollow interior of hub 302 is provided having a funnel shape portion 310 and a straight central longitudinal axis extending from the first end 302a to the second end 302b. Thus hub 302 is referred to as a straight hub.

Needle assembly 300 further includes a needle shaft portion formed of a high hardness metal, the needle shaft portion having a first straight end 305 rigidly affixed to a portion of the hollow interior of the straight hub 302, the first straight end of the needle shaft portion having an embedded section having a length L1 of about 0.375″ which extends at least partially through the hollow interior of the straight hub with at least a portion of the embedded section forming a friction fit within the straight hub portion and an exposed section extending distally beyond the second end of the straight hub, the exposed section having a length of about 2.5″ and an outside diameter of about 18 gauge and the exposed section of the needle shaft portion having a bent shaft portion 306 proximate the second end 302b of straight hub 302 and a straight shaft portion distal of the bent shaft portion with the bent shaft portion forming an angle of about 30 degrees from a central longitudinal axis of the straight hub 302. This particular configuration allows the straight shaft portion of the needle to be inserted into a subclavian vein without a body of a patient interfering with movement or position of the huh while the needle is inserted. For reasons which will become apparent from the description herein below, it is important that the inner diameter throughout the bent portion of the needle be substantially similar to (and ideally, identical to) the inner diameter of the straight shaft portion.

The needle 300 thus has a straight, distal portion 304 that is inserted into the patient. In an embodiment, the needle 300 has a bend 306 between the straight portion 304 and the hub 302. In one exemplary embodiment, the bent shaft portion follows a radius of about 0.8″.

The needle 300 may be partially inserted into the hub and affixed in place with an adhesive. In an embodiment, the portion of the needle 300 that extends at least partially into the hub is about 0.375 inches long.

There is an opening 308 in the proximal end of the hub where a practitioner can insert a guide wire, attach a syringe, etc., as will be discussed below. The opening can have a tapered or funneled internal section 310 that can guide a guide wire into the needle.

The interior of the needle portion (i.e. the surface of the interior walls of the needle), the interior of the hub 302 (i.e. the surface of the interior walls of the hub) form a lumen 312 having a proximal lumen aperture 308 (also referred to as a lumen opening 308) through which a guide wire can be inserted. In order for the guide wire to move smoothly through the lumen, the inner diameter of the lumen is substantially the same distal to the funnel portion 310 along the length of the needle. In particular, the inner diameter of the bent portion 306 is substantially the same as the inner diameter of the straight portion 304 so that a guide wire pushed through the needle can move smoothly through the lumen. That is, a mechanical resistance presented to the guide wire as it travels through the lumen 312 remains substantially the same as the guide wire travels through the entire length of the lumen (i.e. the mechanical resistance presented to the guide wire by the inner walls of the needle which form the lumen is substantially the same in the needle region before the bend, after the bend and in the bent region).

One skilled in the art will recognize that a conventional needle have a lumen extending there through (i.e. a conventional straight needle having a lumen) which is bent using a conventional mechanical bending technique may result in the inner needle walls (i.e. the interior surface of the inner needle walls which form the lumen) becoming partially or completely collapsed at or near the point or region of the bend (i.e. there is a collapsed lumen portion). This collapsed lumen portion makes it difficult or impossible for a guide wire to be inserted through the entire length of the lumen due to the collapsed portion partially or completely occluding the lumen.

As recognized in accordance with one aspect of the concepts sought to be protected herein, a clinician inserting the guide wire through the lumen must be able to feel resistance when the guide wire exits the distal end of the lumen and comes in contact with structures within the patient's body (e.g. a wall of a vein). As used herein, the term clinician includes but is not limited to a surgeon, physician's assistant (PA) or other medical practitioner or person performing or assisting in a medical procedure. If the clinician cannot properly feel a change in resistance of the guide wire, the clinician could inadvertently puncture the wall of the vein.

A straight needle that is bent using conventional mechanical bending techniques (and thus results in a collapsed lumen portion) will not allow a surgeon to precisely feel or detect such resistance caused by contact with a body part since the bent potion of the needle serves as a region of mechanical resistance of the guide wire.

In one exemplary embodiment, the needle is made from an 18 Gauge XTW (extra thin wall having an inside diameter in the range of 0.0410″ to 0.0430″) stainless steel hypodermic needle stock. The bent portion 306 may be bent between about 15 degrees and about 60 degrees from the centerline of the hub 302. In an embodiment, the needle may be bent at 30 degrees (substantially 30 degrees) or 45 degrees from the centerline of the huh 302.

The straight needle portion 304 may be between about two and about three inches long. In one exemplary embodiment, the straight needle portion 304 is 2.5 inches long. It has been discovered that a straight needle portion 304 of this length coupled with a bend radius (also referred to herein as a radius of curvature) of 0.8 inches to a bend angle of 30 degrees will allow the needle to be placed for insertion into the subclavian vein without substantial interference by the shoulder of the patient.

The lumen 312 may have an interior diameter between about 0.03 inches and about 0.04 inches. As noted above, lumen 312 may widen into a funnel shape inside hub 302 so that a guide wire inserted into opening 308 will be directed into lumen 312. In other embodiments, the inner diameter of the lumen 312 is larger or smaller depending upon the size of the guide wire to be introduced into the patient through the lumen. The inner diameter of lumen 312 may be slightly larger than the outer diameter of the guide wire that is used. For example, if the guide wire has a diameter of 0.032″, then the lumen may have an inner diameter of 0.034″ to accommodate the guide wire without causing friction or mechanical resistance, such that the guide wire passes substantially smoothly through the lumen 312. In an embodiment, if the inner diameter of the straight portion 304 of the needle is about 0.034″, then the inner diameter of the bent portion 306 may also be about 0.034″ so that the guide wire does not experience any substantial mechanical resistance when passing through the bent portion (ideally, the mechanical resistance presented to the guide wire by the lumen walls is the same along the entire length of the lumen).

The guide wire that passes through lumen 312 may be a flexible, coiled spring wire. Although the guide wire is flexible, if it is bent at too great an angle it may not be able to slide smoothly through lumen 312. Thus, the angle of the bend 306 should be selected sufficiently small so as to allow the guide wire to pass through the lumen with a consistent (and ideally minimal) resistance while at the same time being a sufficiently large angle so as to allow a clinician to insert the needle into a patient without interference from the patient's shoulder.

In addition, if the bend in the needle is too sharp (i.e. if the radius of curvature of the bend is too small) then the guide wire may not be able to slide smoothly through lumen 312. Thus, the bend should have a radius of curvature sufficiently large so that the bend is gentle enough for the guide wire to past through without significant resistance.

Referring to FIG. 4, a needle assembly 400 includes a straight needle portion 402 and a bent or curved hub 404. The proximal end of the hub 404 includes an opening 406. A syringe can be attached to opening 406 and/or a guide wire can be inserted into opening 406. A lumen 408 is formed by the opening, the inner chamber of the hub 404, and the inner pathway formed by needle 402. The inner chamber of hub 404 may have a funnel shape that guides the guide wire into needle 402 when the guide wire is inserted into opening 406.

In an embodiment, the inner diameter of bent section 408 is substantially the same as the inner diameter of straight needle section 402, so that a guide wire can pass through bent section 408 smoothly and with little resistance. In another embodiment, and as shown in FIG. 4, the inner diameter of bent section 408 may be larger than the inner diameter of needle 402, and may form at least part of the funnel shape of the inner chamber of the hub 404.

The needle hubs shown in FIGS. 3 and 4 can be provided having a taper (or funnel shape) along a length thereof. In one exemplary embodiment, needle hubs are equipped with a 6% taper (commonly trade marked as Luer-Lok™) for connection to a syringe. The needle hubs can also be equipped with other types of connections for connecting to syringes, medical tubing, medical test equipment, other medical devices, etc.

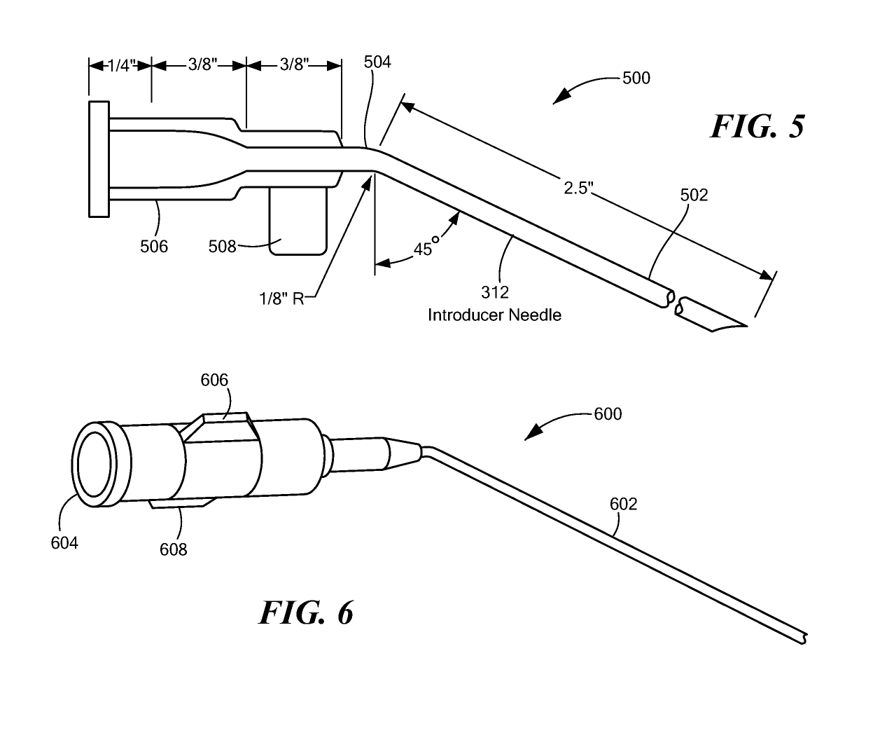

Referring to FIG. 5, a needle assembly 500 includes a straight needle portion 502, a bent portion 504 and a hub 506. The needle assembly 500 may be the same as or similar to needle assembly 200 in FIG. 2 or needle assembly 300 in FIG. 3.

In an embodiment, hub 506 includes an extension 508. The extension 508 is adjacent to the bent section 504 and extends parallel to a plane defined by the central axis of the hub 506 and the straight needle portion 502. Extension 508 may be positioned on the inside angle of bent portion 504. In other words, extension 508 is positioned on the “bottom” of hub, as shown in FIG. 5. In other embodiments, extension 508 can positioned on the outside angle of bent portion 504 (i.e. on the “top” of the hub 506 as shown in FIG. 5). Extension 508 can also extend perpendicularly to the plane defined by the central axis of the hub 506 and the straight needle portion 504. In other words, extension 508 can extend into or out of the page as shown in FIG. 5. In other embodiments, extension 508 can extend at any other angle relative to the plane defined by the centerline of the hub 506 and the needle 502.

The extension 508 may act as a handle that a clinician can hold during a surgical procedure. The clinician may hold the extension 508 between a finger (or fingers) and thumb to stabilize the needle assembly 500 during insertion, when inserting the guide wire, when attaching and detaching a syringe, or during other surgical events. Extension 508 may provide leverage so that the clinician can hold the needle assembly 500 still and prevent it from rotating, for example, when a syringe is attached to or removed from the huh or when a guide wire is inserted through the needle.

Referring to FIG. 6, a needle assembly 600 may be the same as or similar to needle assemblies described above. Needle assembly 600 includes a bent needle portion 602 and a hub 604. The hub 604 includes one or more extensions 606 and 608 that extend from the hub 604, in a direction perpendicular to the plane formed by the needle portion 602 and the centerline of the hub 604. The extensions 606 and 608 may provide a handle or gripping area for a clinician using the needle assembly 600.

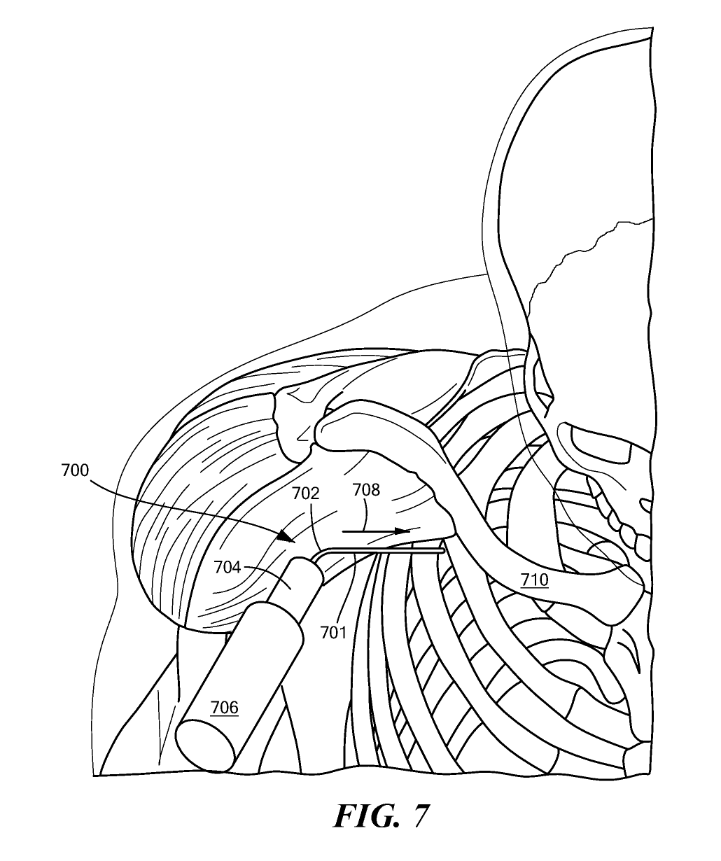

Referring to FIG. 7, a bent needle assembly 700 is shown in operation. The needle assembly 700 may be the same as or similar to bent needle assemblies as described above, and may include a straight needle portion 701, a bent needle portion 702, and a hub 704. If desired, a syringe 706 can be attached to the proximal end of the hub as shown.

When used, with the patient in a supine position, a clinician can align the straight needle portion 701 perpendicularly to the floor and pointed medially toward the sternal notch. The hub 704 and syringe 706 can be positioned so that they are angled away from the patient's shoulder (e.g. away from the floor), so that the patient's shoulder does not interfere with placement of the needle. The needle may then be inserted into the patient's subclavian vein, just below the clavicle 710, in the direction shown by arrow 708.

Even with the patient's shoulder in a normal position, the bent needle can be positioned so that insertion is performed in a direction substantially parallel to the floor. Inserting the needle in this manner allows the needle to stay very close to the surface of the patient's chest without angling the needle subcutaneously toward the upper rib cage and lung (i.e. without angling the needle toward the floor). Because the hub 704 and syringe 706 are angled away from the shoulder, a scapula wedge, rolled towel, or other mechanism that moves the patient's shoulder out of the way is not necessary to insert the needle in a safe manner, parallel to the floor, that minimizes potential complications.

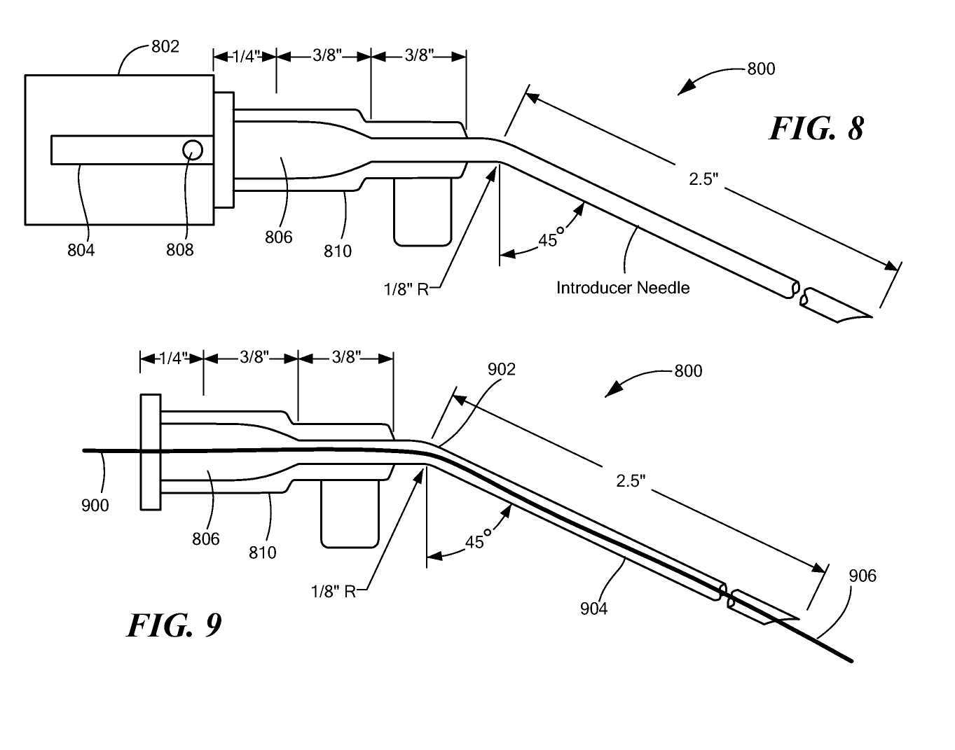

Referring now to FIG. 8, a needle assembly 800 is shown attached to a syringe 802 in order to allow the clinician to test whether the needle has been successfully inserted into the subclavian vein. The syringe 802 may be a so-called Raulerson syringe having an internal lumen that can mate with lumen 806 of the needle assembly 800. A plunger of the syringe 802 (not shown) can create a vacuum force within chamber 806 of the syringe 802. When the needle penetrates the vein, the vacuum force will draw blood through a hole 808 in the lumen 804. When blood expresses from the hole 808, the needle has penetrated the vein successfully. The blood can also act to lubricate the lumen to further reduce resistance when a guide wire is placed through the lumen.

Referring now to FIG. 9, once the needle has penetrated the vein, the clinician can remove the syringe 802 from hub 810 and a guide wire 900 can be advanced through lumen 806 into the patient's vein. As noted above, the guide wire will not experience substantial resistance due to the bent portion 902 because the interior diameter of the bent portion 902 may be substantially similar in size to the inner diameter of the straight needle portion 904. This can help to avoid complications by allowing the clinician to move the guide wire in and out and use the tip 906 to “feel” whether the needle placement is correct. For example, if the guide wire 900 can move into the vein freely, then the needle has been placed correctly. However, if the tip 906 of the guide wire 900 comes in contact with the wall of the vein or another object, the needle may not have been placed correctly or there may be some other complication with the surgery. If bent portion 902 had a smaller inner diameter that impeded movement of the guide write 900, it could hamper the clinician's ability to use the guide wire to feel inside the patient's body.

In an embodiment, the syringe 802 in FIG. 8 may be permanently attached to the needle assembly 800. In this case, lumen 806 may extend though the syringe so that a guide wire can be inserted through the syringe, hub, and needle portion.

Once the guide wire is inserted into the patient's vein, the needle assembly 800 can be removed. The guide wire can then be used to insert a medicinal port (e.g. a subclavian catheter) into the vein, which can be used to administer drugs directly into the bloodstream or to perform other surgical procedures.

Manufacturing

Simply bending a surgical needle will result in the bent portion having a smaller inner diameter than the remainder of the needle. This is due to plastic deformation of the metal at the bend site. In order to manufacture a bent needle having a bent portion with an inner diameter that is substantially similar (or the same as) the inner diameter of the remainder of the needle, a force must be applied to the needle during the bending process.

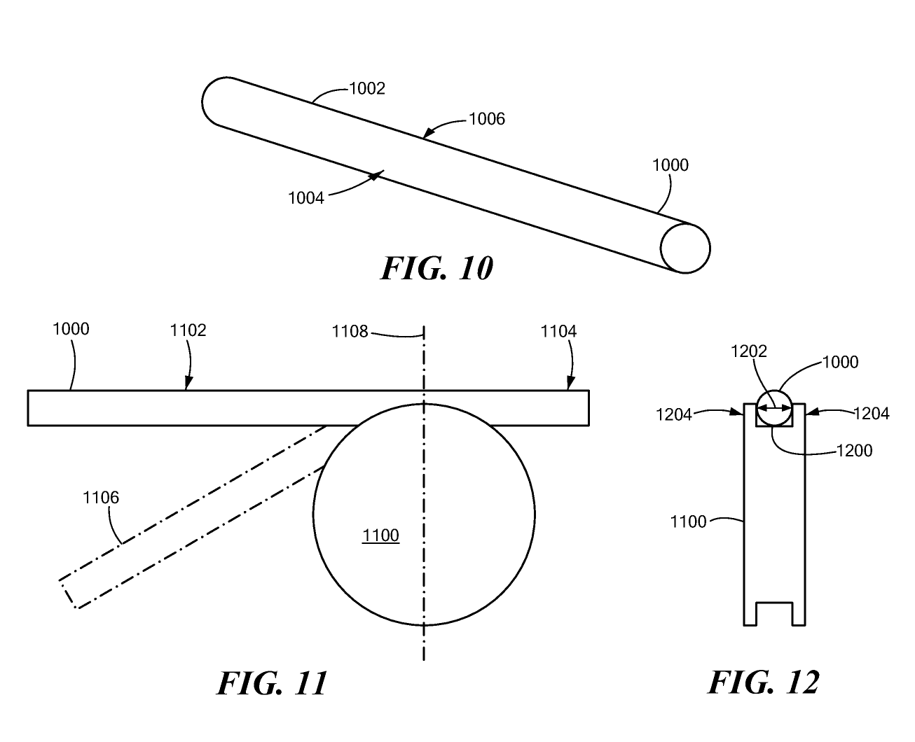

Referring to FIG. 10, a straight needle shaft 1000 is shown prior to bending. During the bending process, the needle will be bent at location 1002. So that the tubular shape of needle shaft 1000 does not collapse at location 1002, a force can be applied to the opposite “sides” of needle shaft 1000 as shown by arrows 1004 and 1006. Applying the force 1004 and 1006 will prevent the sides of needle shaft 1000 from expanding outward during bending, thus preventing the needle shaft 1000 from collapsing at location 1002.

Referring to FIG. 11, in order to apply the force 1004 and 1006, a bending jig can be used. FIG. 11 shows a side view of an embodiment of one such bending jig. In one embodiment, the bending jig includes a wheel 1100 around which the needle shaft can be bent. The needle can be placed in a groove around the edge of the wheel (see FIG. 12 below), and a force can be applied in the direction of arrow 1102 and/or 1104 to bend the needle shaft 1000 around the wheel 1100. In an embodiment, dotted line 1106 shows a final position of the needle shaft 1000 after it has been bent.

The diameter of the wheel 1100 can be adjusted to define the radius of curvature of the bend. In an embodiment, the radius of curvature may be 0.125 inches. In other embodiments, the radius of curvature may vary from 0.125 inches to 1 inch.

FIG. 12 is a cross sectional view of wheel 1100 and needle shaft 1000 shown at dotted line 1108. Wheel 1100 includes a groove 1200 into which needle 1000 can be placed during bending. The groove has a width that is the same as the diameter of needle shaft 1000. In an embodiment, the groove can have a width that is slightly larger than the needle shaft 1000 so that the needle shaft 1000 can be inserted into and removed from the groove 1200 easily.

As the needle is bent, the sides of the needle shaft 1000 will attempt to expand in the direction shown by arrow 1202. However, the side walls of groove 1200 will provide an equal and opposite force (shown by arrows 1204) to the needle shaft 1000 to prevent the sides of the needle from expanding and prevent the needle shaft 1000 from collapsing at the site of the bend. Because needle shaft 1000 cannot collapse during the bending process, the bent portion of the resulting bent needle will have an inner diameter that is the same as or substantially similar to the inner diameter of the remainder of the needle.

Although shown as having a rectangular shaped groove 1200, wheel 1100 can also have a rounded groove to seat the needle shaft 1000, or any other shape, so long as groove 1200 can apply force 1204 to prevent the needle shaft 1000 from collapsing during the bending process.

In an embodiment, the bent needle can have the following measurements and parameters: Angle between hub axis and needle axis—135-degrees, +/−5, degrees; Length of bent needle −2.5″, −0″ or +0.5″ Size of bent needle—18 Gauge XTW with or without an extra thin wall; Needle inside diameter—0.042″, +/−0.001″; Needle outside diameter—0.050″, +/−0.0005″; Needle material—stainless steel, hypodermic needle stock.

Having described preferred embodiments, which serve to illustrate various concepts, structures and techniques, which are the subject of this patent, it will now become apparent to those of ordinary skill in the art that other embodiments incorporating these concepts, structures and techniques may be used. Accordingly, it is submitted that that scope of the patent should not be limited to the described embodiments but rather should be limited only by the spirit and scope of the following claims. All references cited herein are hereby incorporated herein by reference in their entirety.

What is claimed is:

1. A needle for subclavian center line catheter placement, the needle comprising:
a proximal needle portion corresponding to a straight hub and formed of a rigid material, the straight hub having an outside diameter, a hollow interior, and a length, and having a first end configured to be coupled to a syringe and a second end; and
a needle shaft portion formed of a high hardness metal, said needle shaft portion having a first straight end rigidly affixed to a portion of the hollow interior of the straight hub, the first straight end of the needle shaft portion having:
an embedded section having a length of about 0.375″ which extends at least partially through the hollow interior of the straight hub with at least a portion of the embedded section forming a friction fit within the straight hub portion; and
an exposed section extending distally beyond the second end of the straight hub, the exposed section having a length of about 2.5″ and an outside diameter of about 18 gauge and the exposed section of the needle shaft portion having a bent shaft portion proximate the second end of the straight hub and a straight shaft portion distal of the bent shaft portion with the bent shaft portion forming an angle of about 30 degrees from a central longitudinal axis of the straight hub such that the straight shaft portion of the needle may be inserted into a subclavian vein without a body of a patient interfering with movement or position of the hub
wherein the inner diameter of the bent portion is substantially similar to the inner diameter of the straight shaft portion.

2. The needle of claim 2 wherein the bent shaft portion follows a radius of about 0.8″.

3. A needle for subclavian center line catheter placement, the needle comprising:
a proximal needle portion corresponding to a straight hub and formed of a rigid material, the straight hub having an outside diameter, a hollow interior, and a length, and having a first end configured to be coupled to a syringe and a second end; and
a needle shaft portion formed of a high hardness metal, said needle shaft portion having a first straight end rigidly affixed to a portion of the hollow interior of the straight hub, the first straight end of the needle shaft portion having:
an embedded section having a length which extends at least partially through the hollow interior of the straight hub with at least a portion of the embedded section forming a friction fit within the straight hub portion; and
an exposed section extending distally beyond the second end of the straight hub, the exposed section having a length and an outside diameter and the exposed section of the needle shaft portion having a bent shaft portion proximate the second end of the straight hub and a straight shaft portion distal of the bent shaft portion with the bent shaft portion forming an angle sufficient to allow the straight shaft portion of the needle to be inserted into a subclavian vein without a body of a patient interfering with movement or position of the hub;
wherein the bent shaft portion has an interior diameter substantially similar to an inner diameter of the straight shaft portion.

4. The needle of claim 3 wherein portion of the huh includes a curved portion that extends along an outer surface of the bent portion and at least partially covers the bent portion.

5. The needle of claim 3 wherein the hub includes a tapered internal channel to allow a lumen to be extended through the hub and needle into the subclavian vein.

6. The needle of claim 3 wherein the needle shaft bent portion is located within an inch of the proximal end of the needle shaft.

7. The needle of claim 6 wherein the shaft is bent at an angle of between about 15 and about 50 degrees.

8. The needle of claim 7 wherein the shaft is bent at an angle of about 30 degrees.

9. The needle of claim 8 wherein the bend has radius of curvature of about 0.125 inches.

10. The method of claim 9 wherein the straight shaft portion has a length between about 2 and about 3 inches.

11. The method of claim 10 wherein the straight shaft portion has a length of about 2.5 inches.

12. The needle of claim 11 wherein the needle shaft has an inside diameter of between 0.0410 and 0.0430 inches.

13. The needle of claim 12 wherein the needle shaft is an 18 gage extra thin wall needle.

14. The needle of claim 15 wherein the hub comprises at least one tab.

15. The needle of claim 14 wherein the hub extents along an axis in a plane, the shaft is bent at an angle from the axis to lie in the same plane, and the tab extends perpendicular to the plane.

16. The needle of claim 15 wherein the huh and needle shaft are formed as a single piece.

17. The needle of claim 15 further comprising a syringe permanently attached to a proximal end of the hub.
